(19) United States
(12) Patent Application Publication
(10) Pub. No.: US 2010/0316741 A1
(43) Pub. Date: Dec. 16, 2010

(54) COMPOSITION COMPRISING THE EXTRACT OF CRUDE DRUG COMPLEX FOR STIMULATING BONE GROWTH

(75) Inventors: Hocheol Kim, Seoul (KR); Mi-Yeon Kim, Seoul (KR); Young Mi Park, Seoul (KR); Yun Tai Kim, Incheon (KR); Zhen Hua Jin, Seoul (KR); Dong Wook Lim, Seoul (KR); WonHong Gaugh, Seoul (KR); Youngmin Bu, Seoul (KR)

Correspondence Address: GREENLEE SULLIVAN P.C., 4875 PEARL EAST CIRCLE, SUITE 200, BOULDER, CO, 80301, US

(73) Assignee: Kyunghee University-Industry Cooperation Foundation, Youngin-si, Gyeonggi-do (KR)

(21) Appl. No.: 12/518,010

(22) PCT Filed: Dec. 10, 2007
(86) PCT No.: PCT/KR2007/006406

§ 371 Date: Jun. 5, 2009

(30) Foreign Application Priority Data
Dec. 8, 2006 (KR) 10-2006-0124743

(51) Int. Cl. A61K 36/254; A61P 19/00

(52) U.S. Cl. 424/728

(57) ABSTRACT

The present invention relates to a composition for stimulating bone growth containing the extract of crude drug complex as an active ingredient. The extract of crude drug complex comprising Dipsaci radix, Astragalus membranaceus and Acanthopanax senticosus of the present invention stimulates bone growth, so that it can be effectively used for the composition and health food for stimulating bone growth.

### TECHNICAL FIELD

The present invention relates to a composition for stimulating bone growth comprising the extract of crude drug complex as an active ingredient, more precisely a composition for stimulating bone growth comprising the extract of crude drug complex composed of Dipsaci radix, Astragalus membranaceus and Acanthopanax senticosus.

### BACKGROUND ART

Growth indicates the increase of height, which is stimulated by nutrition and growth hormones. The nervous system including the brain secreting growth hormone is fast growing in childhood and since then, the growth becomes slow. The growth continues until the age of puberty in most human. So, if nutrition is not taken enough particularly from childhood to the age of puberty, the full growth cannot be expected. For nutritional balance, numbers of growth stimulating health foods have been sold. However, these health foods are prepared simply by combining many components for stimulating growth and the effect is not satisfactory.

The growth of the length of long bone determines the height and bone structure, which is regulated by a specific mechanism. In particular, the growth of the epiphyseal growth plate of long bone is the most important index for the growth of bone length. The growth plate is composed of proliferation zone involved in the proliferation of chondrocytes, maturation zone involved in the growth of chondrocytes and hypertrophic zone involved in the hypertrophy of chondrocytes, and the interrelation among those zones leads to the growth of the length of long bone (Loveridge, N, J. Anim. Sci. 77, 190-196, 1999).

The growth hormone has been used for the treatment of dwarfism. Since the late 1985, the growth hormone prepared by genetic recombination has been on market, suggesting that the mass production and supply of such growth hormone has been accomplished. Besides, there was no risk of getting a disease by taking the growth hormone. So, the growth hormone produced by genetic recombination has been recently used as a therapeutic agent for treating diseases caused by deficiency of growth hormone. GHRP-6 (growth hormone releasing peptide-6, His-D-Trp-Ala-Trp-D-Phe-Lys-NH2), the growth hormone secretion stimulator, which is composed of 6 amino acids having growth hormone secreting activity has been developed based on enkephalin (Bowers C Y. et al., Endocrinology, 114, 1537-45, 1984). According to the previous report, this drug can be absorbed when it is oral-administered, even if the absorption rate is very low (Noriko I. et al., Life Science, 47, 29-36, 1990). The studies continued and other growth hormone secretion stimulators, GHRP-1 (Ala-His-D-β-Nal-Ala-Trp-D-Phe-Lys-NH2) and GHRP-2 (D-Ala-D-β-Nal-Ala-Trp-D-Phe-Lys-NH2) composed of 6 modified peptides, have been developed. Even if the in vivo activity of these drugs was excellent, the absorption rate according to the oral administration was only 2-3%, indicating that it is still limited for oral administration.

Dipsaci radix is the root of Dipsacus asper Wall that is a perennial herb belonging to Dipsacaceae. The major components are succinic acid, betonicine, shanzhiside methyl ester, akebia saponin D and daucosterol. According to the traditional oriental medicine, Dipsaci radix has been used as a drug that has the effect of invigorating liver and kidney, strengthening muscle and bone, forming muscle and bone and treating waist and kidney diseases; in other words it has been used as a drug for making bones and muscles strong by invigorating the body particularly when bones and muscles were weakened. So, a method for separation and purification of asperosaponin H1, a kind of glycoalkaloid, derivative that is one of the major components of Dipsaci radix and has the activity of stimulating the secretion of growth hormone is described in Korean Patent No. 10-0436219. There are references illustrating the effect of Dipsaci radix (Yu, et al., Effect of the Physiologically Active Compounds in Dipsaci radix on Cell Cycle Regulation in Human Gingival Fibroblasts, Journal of Korean Academy of Periodontology, 35(1), 87, 2005; Kim, et al., The Effects of Dichloromethane fraction of Dipsaci radix (DFPR) on differentiation of Mouse Calvarial Cell, Journal of Korean Academy of Periodontology, 34(4), 791, 2004; Ahn, et al., Effects of the Dipsaci radix on the Aged ovariectomized Rat Model of Postmenopausal Osteoporosis, Journal of Korea Association of Herbology, 9(1), 181, 1994; Chun-Hsu Yao, Effects of Dipsaci radix on the osteoblast differentiation, J Biomed Master Res Part B: Appl Biomaster 75B: 277-88, 2005). So, it is presumed that the effect of Dipsaci radix on the growth is achieved by stimulating the osteoblast differentiation to induce bone growth. However, the effect of Dipsaci radix on the growth plate has not been exposed, yet.

Astragalus membranaceus is the root, whose periderm is almost peeled, of the perennial herb Astragalus membranceus Bunge belonging to Leguminosae. The major components are sucrose, glucoronic acid, different kinds of amino acids, bitter substance, mucosubstance, choline, betaine and folic acid. According to the traditional oriental medicine, Astragalus membranaceus has the effect of strengthening the body, for example it helps the treatment of weak body, sweating, furuncurus, dipsesis, abdominal pain, kidney weakness, ear pus, diabetes, pain, loss of taste, lack of appetite, hemorrhoid, red eye, malaria, proctoptosis, metrorrhagia, colporrhea, menstrual irregularity, prenatal/postbirth symptoms, discharge of phlegm, headache, petrified lung, heart burning, dysentery, juvenile diseases, etc. There are references that explain the effect of Astragalus membranaceus on hematopoiesis (Ma R. et al., J Tradit Chin Med. September; 3(3):199-204, 1983), on antioxidation (Li, Chun-Ying., J. Agr. Sci., Inst. Agr. Sci. Kangwon Nat. Univ., 15:103, 2004), on the osteoblast differentiation in rabbit (Xu C J. et al., Zhong Nan Da Xue xue Bao Yi Xue Ban, August; 29(4):489-91, 2004), on the prevention of osteoporosis of ovariectomized rat (Kim C. et al., Arch Pharm Res. November; 26(11):917-924, 2003). However, the effect of Astragalus membranaceus on the growth plate has not been exposed, yet.

Acanthopanax senticosus indicates the dried root, rhizome and bark of the deciduous shrub Acanthopanax senticosus (RUPR. et MAXIM) HARMS belonging to Araliaceae. It contains such glycosides as carotene, ligustrin, 7-methyl-6,8-dimethylcoumarineglycoside, galactoside, syringaresinol di-o-beta-D-glucoside and caryophyllen and chlorogenic acid, flavone, refined oil and polysaccharide as major components. The pharmacological actions of Acanthopanax senticosus are sedation, anti--stress, immune enhancement, smooth muscle relaxation, and anti-inflammation (State Administration of Traditional Chinese Medicine: Zhonghuabencao, Shanghai, Shanghai Scientific and technical Publishers 1998). In addition, Acanthopanax senticosus is also known to have the effect of strengthening spleen and sprit, invigorating kidney, improving weak waist, invigorating, relaxation and helping blood circulation without interruption, so that it has been widely used for the treatment of exhaustion resulted from weakening of lung and spleen, Weakness of lower limbs and back caused by lack of appetite and weak spleen and kidney, poor blood circulation resulted from the loss of stamina, weak heart and spleen, inappetence, sleeplessness or bad dreaming, weak muscles and bones in child, Dropsical leg, and rheumatoid arthritis (State Administration of Traditional Chinese Medicine: Zhonghuabencao, Shanghai, Shanghai Scientific and technical Publishers, 1998). Acanthopanax senticosus has the effect of improving tonic with invigorating mentally and physically and the preventive effect on disease owing to the capability of increasing non-specific resistance (Wagner, 1985) as well as the activity of lowering blood sugar and inhibiting cancer and hypotension. Unlike ginseng, Acanthopanax senticosus does not have such side effect as insomnia since it does not make people nervous (Loydia, Vol. 32. 1, 1969). Acanthopanax senticosus has protective effect against the toxicity caused by parathion (Ferguson P. W. et al., 1984) and irradiation (Miyanomae T et al., 1988), and has anti-coagulation activity (Yun-Choi H. S. et al., 1987) and has the effect of lowering blood lipid level (Sui D. Y. et al., 1994). Among the components of Acanthopanax senticosus, acanthoic acid inhibits platelet coagulation, inflammation, and the release of superoxideanion (Kang H. S. et al., Cell Immuno. Vol. 170, No. 2, p 212, 1996). In the meantime, chlorogenic acid and syringaresinol di-o-beta-D-glucoside reduce the risk of gastric ulcer caused by cool bath stress (Fujikawa T. et al., 1996). Eleutheroside B inhibits inflammatory COX and diterpene molecules (pinoresinol, sesamin, etc) have the equal activity to the above (Bull K. H. Pharma. Sci. vol. 18, p 43, 1990). As steroid hormone inhibits PLA2 (phospholipase A2), steroids such as β-sitosterol has the same activity so that they prevent arachidonic acid from being separated from phospholipids of cell membrane (Korean Patent Publication No. 2000-9820). Based on the disclosed pharmacological actions of Acanthopanax senticosus as explained hereinbefore, the studies have been focused on the different uses of Acanthopanax senticosus. Korean Patent Publication No. 2000-9820 describes the treatment of acne and whelk using Acanthopanax senticosus. Korean Patent Publication No. 2000-74868 describes the treatment effect of Acanthopanax senticosus on erectile dysfunction. Korean Patent Publication No. 160402 describes that Acanthopanax senticosus is used as an active ingredient for a composition for anti-stress. And the present inventors previously explained that the extract of Acanthopanax senticosus has the neuronal protective effect in Korean Patent Application No. 2001-17459.

The present inventors had screened natural substances to overcome the disadvantages of growth hormone commercialized for stimulating growth such as high costs, low absorption rate and side effects. And the inventors completed this invention by confirming that the extract of crude drug complex comprising Dipsaci radix, Astragalus membranaceus and Acanthopanax senticosus had better growth stimulating effect than growth hormone hypodermically injected.

### DISCLOSURE

### Technical Problem

It is an object of the present invention to provide a composition for stimulating bone growth comprising the extract of the herb mixture composed of Dipsaci radix, Astragalus membranaceus and Acanthopanax senticosus as an active ingredient.

### Technical Solution

To achieve the above object, the present invention provides a composition for stimulating bone growth comprising the extract of crude drug complex composed of Dipsaci radix, Astragalus membranaceus and Acanthopanax senticosus as an active ingredient.

The present invention also provides health food for stimulating bone growth comprising the above composition as an active ingredient.

The present invention further provides a method for stimulating bone growth comprising the step of administering the effective dose of the composition to a subject.

### Advantageous Effect

The extract of crude drug complex comprising Dipsaci radix, Astragalus membranaceus and Acanthopanax senticosus of the present is so effective in stimulating growth that the extract can not only be added to a composition for stimulating bone growth but also :be utilized as health food.

### DESCRIPTION OF DRAWINGS

The application of the preferred embodiments of the present invention is best understood with reference to the accompanying drawings, wherein:

FIG. 1 is a photograph of fluorescent microscope showing the luminescence developed by deposition of tetracycline in the epiphyseal growth plate of the tibia of hind limb of a rat:

A: saline treated group; B: growth hormone (20 μg/kg) treated group; C: crude drug complex extract (100 mg/kg) treated group (example 1); D: crude drug complex extract (100 mg/kg) treated group (example 2) and E: crude drug complex (100 mg/kg) treated group (example 3).

FIG. 2 is a graph illustrating the interval between the growth plate and luminescent line observed after 48 hours from the tetracycline injection to the rat:

*: P<0.01; and ***: P<0.001.

FIG. 3 is a graph illustrating the growth rate of the rat:

*: P<0.01; and ***: P<0.001.

### MODE FOR INVENTION

Hereinafter, the present invention is described in detail.

The present invention provides a composition for stimulating bone growth comprising the extract of crude drug complex as an active ingredient.

The extract of crude drug complex was prepared as follows. Dipsaci radix, Astragalus membranaceus and Acanthopanax senticosus (Kyung-Dong Market, Seoul, Korea) were dried and pulverized. The dried sample was added into a solvent selected from water, C₁-C₄ low alcohol such as ethanol and methanol, and a mixed solvent thereof (the ratio of water:alcohol is 1:0.1-1:10, preferably 1:0.2-1:5) of 1-20 times the weight of the dried sample, more preferably 5-15 times the weight of the dried sample, followed by extraction at room temperature for 1-24 hours, preferably for 5-15 hours and more preferably for 6 hours. The extraction method can be exemplified by hot water extraction, enfleurage, reflux extraction and ultrasonic extraction. But it is more preferred to perform extraction by reflux extraction followed by vacuum concentration to obtain the crude extract of the present invention.

The ratio of constituents in the crude drug complex of the invention is as follows; Dipsaci radix 1-5 weight part, Astragalus membranaceus 1-3 weight part and Acanthopanax senticosus 1-5 weight part, more preferably Dipsaci radix 1-2 weight part, Acanthopanax senticosus 1-3 weight part and Acanthopanax senticosus 3-5 weight part, and most preferably Dipsaci radix 1.5 weight part, Astragalus membranaceus 3 weight part and Acanthopanax senticosus 5 weight part.

To measure the growth stimulated by the extract of crude drug complex of the invention, rats were divided into three groups of negative control group, experimental group and positive control group. To the negative control group, saline was orally administered twice a day for 4 days. To the experimental group, the extract of crude drug complex (see Examples 1-5 and Table 1) or the extract of each herb medicine (see Comparative Examples 1-3 and Table 2) was orally administered likewise. To the positive control, growth hormone was hypodermically injected. The growth was investigated after tetracycline was deposited on the growth plate by measuring the extended length of tibia, which was determined by measuring the interval between the growth plate and the luminescent line developed by the deposit of tetracycline. Particularly, on the 3^{rd} day, tetracycline was injected into the abdominal cavity of the rats. On the 5^{th} day, 48 hours later from the tetracycline injection, the rats were anesthetized with ether and sacrificed.

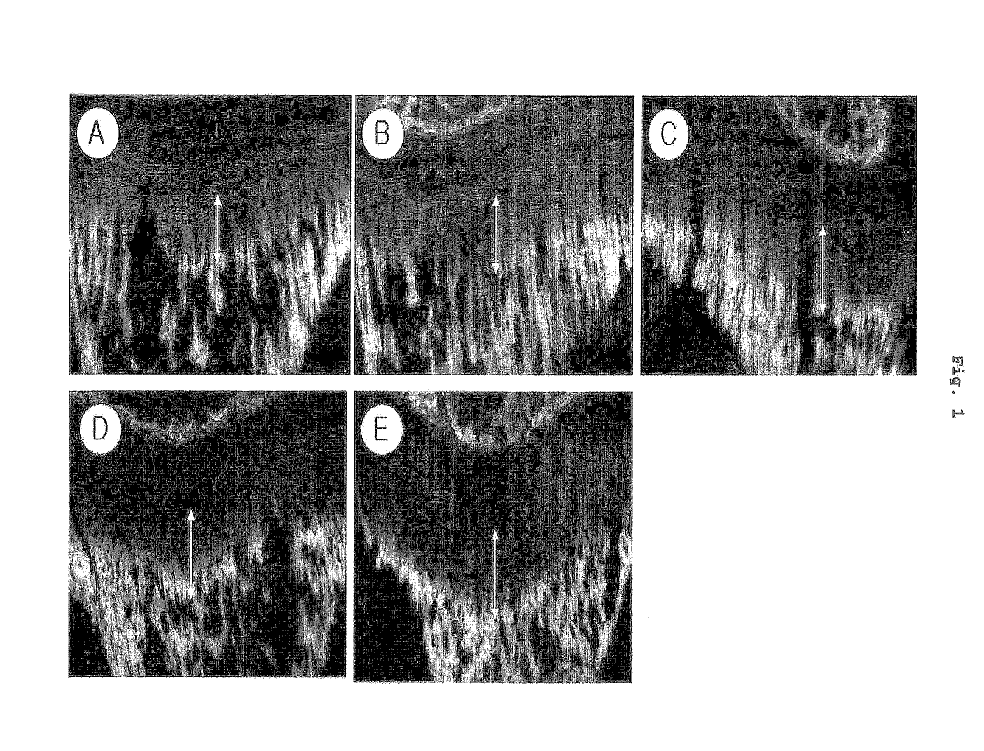

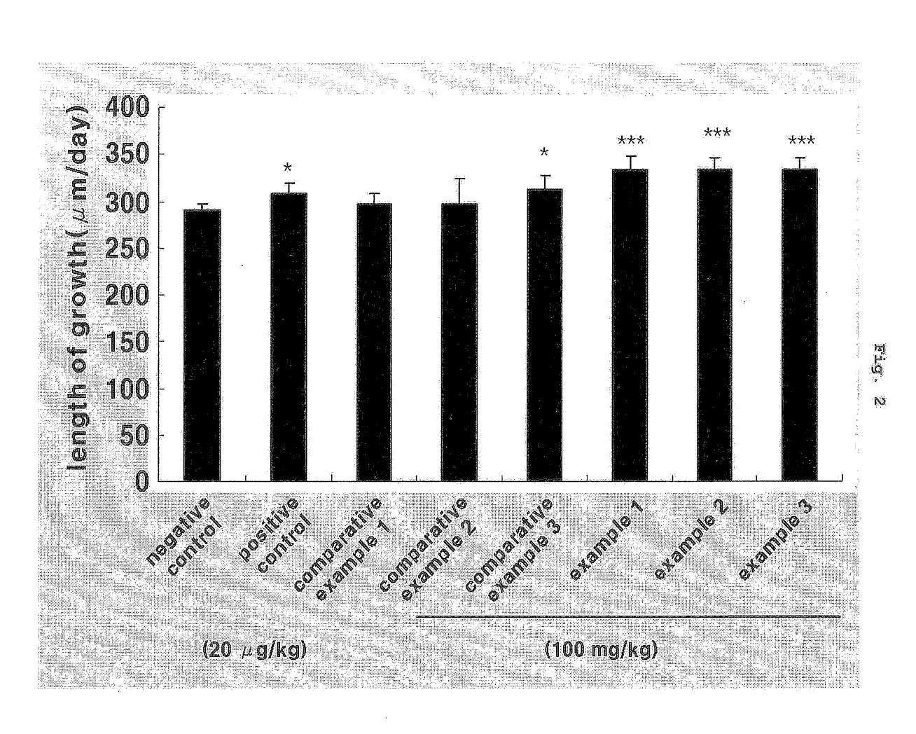

In the rats, the interval between the growth plate and the luminescent line developed by the deposit of tetracycline was photographed by fluorescent micrograph (see FIG. 1) to measure the growth. As a result, the extract of crude drug complex stimulated the bone growth. The bone growth rate of the experimental group treated with the extract of crude drug complex (see Examples 1-5 and Table 1) was statistically higher than those of the negative control group and the group treated with the single extract of each herb (see Comparative Example 1-3 and Table 2) and also higher than that of the positive control group. The bone growth rate of the group treated with the single extract of each herb (see Comparative Example 1-3 and Table 2) was higher than that of the negative control group but lower than that of the positive control (see Table 3 and FIG. 2). * indicates P<0.01, and *** indicates indicates P<0.001. Considering the bone growth rate of the negative control treated with saline as 100%, the bone growth rate of the experimental group treated with the extract of crude drug complex was higher than that of the positive control injected with growth hormone. The growth rate of the group treated with the single extract of each herb (see Comparative Example 1-3 and Table 2) was lower than that of the positive control (see Table 3 and FIG. 2). Therefore, it was confirmed that the bone growth stimulating effect of the extract of crude drug complex was higher than any of the single extract of each herb and growth hormone.

The extract of crude drug complex of the invention was orally administered to mice for toxicity test. As a result, the extract orally administered in this experiment was evaluated to be a safe substance since its estimated LD₅₀ value was much greater than 1,000 mg/kg in mice.

The extract of crude drug complex is very safe and has better growth stimulating effect than growth hormone does, making it as an excellent candidate for a composition for stimulating bone growth.

The composition for stimulating bone growth of the present invention preferably includes the extract of crude drug complex by 0.1-50 weight% for the total weight of the composition, but not always limited thereto. The composition comprising the extract of crude drug complex can additionally include generally used carriers, excipients and diluents.

The pharmaceutically acceptable formulation for administration of the extract of crude drug complex can be pharmaceutically acceptable salts thereof, which can be administered independently or as a mixture with other pharmaceutically active compounds.

The composition of the present invention can be formulated for oral or parenteral administration by mixing with generally used fillers, extenders, binders, wetting agents, disintegrating agents, diluents such as surfactant, or excipients. Possible suitable formulations are oral preparations such as powders, granules, tablets, capsules, suspensions, emulsions, syrups and aerosols, preparations for external use, suppositories and sterile injections. The carriers, excipients and diluents are exemplified by lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil.

Solid formulations for oral administration are tablets, pills, powders, granules and capsules. These solid formulations are prepared by mixing one or more suitable excipients such as starch, calcium carbonate, sucrose or lactose, gelatin, etc. Except for the simple excipients, lubricants, for example magnesium stearate, talc, etc, can be used. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin.

Formulations for parenteral administration are sterilized aqueous solutions, water-insoluble excipients, suspensions, emulsions, lyophilized preparations, suppositories and injections. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerogelatin, etc.

The effective dosage of the crude drug complex of the present invention can be determined according to weight and condition of a patient, severity of a disease, preparation of a drug, administration pathway and time. The effective dosage of the extract of crude drug complex is preferably 0.0001-100 mg/kg per day, and more preferably 0.001-100 mg/kg per day. The administration frequency can be once a day or a few times a day. The above dosage cannot limit the scope of the invention in any way.

The extract of crude drug complex of the present invention can be administered to rats, mice, cattles and mammals including human by various pathways, for example the possible administration pathway can be oral administration, rectal administration, intravenous injection, intramuscular injection, hypodermic injection, intrauterine injection or intracerebroventricular injection.

The extract of crude drug complex of the present invention barely has toxicity or side effects, so that it is a safe composition for long term administration.

The present invention also provides health food for stimulating bone growth comprising the composition of the invention as an active ingredient.

The extract of crude drug complex of the present invention can be added to various grocery foods, dairy products, beverages, gums, teas, vitamin complexes, health foods, etc. The health food containing the extract can be prepared in various formulations such as powders, granules, tablets, capsules or beverages.

The extract of the invention can be added to foods or beverages for the purpose of stimulating bone growth. At this time, the content of the extract in such foods and beverages is 0.01-15 weight % of the total food weight. Particularly, the content of the extract in health beverages is 0.02-5 g for 100 ml of beverages and more preferably 0.3-1 g.

The composition for health beverages of the present invention can additionally include various flavors or natural carbohydrates, etc, like other beverages in addition to the extract of crude drug complex. The natural carbohydrates above can be one of monosaccharides such as glucose and fructose, disaccharides such as maltose and sucrose, polysaccharides such as dextrin and cyclodextrin, and sugar alcohols such as xylitol, sorbitol and erythritol. Besides, natural sweetening agents (thaumatin, stevia extract, for example rebaudioside A, glycyrrhizin, etc.) and synthetic sweetening agents (saccharin, aspartame, etc.) can be included as a sweetening agent. The content of the natural carbohydrate is preferably 1-20 g and more preferably 5-12 g in 100 ml of the composition.

In addition to the ingredients mentioned above, the extract of crude drug complex of the present invention can include in variety of nutrients, vitamins, minerals (electrolytes), flavors including natural flavors and synthetic flavors, coloring agents and extenders (cheese, chocolate, etc.), pectic acid and its salts, alginic acid and its salts, organic acid, protective colloidal viscosifiers, pH regulators, stabilizers, antiseptics, glycerin, alcohols, carbonators which used to be added to soda, etc. The extract of crude drug complex of the present invention can also include natural fruit juice, fruit beverages and/or fruit flesh addable to vegetable beverages. All the mentioned ingredients can be added singly or together. The mixing ratio of those ingredients does not matter in fact, but in general, each can be added by 0.1-20 weight part per 100 weight part of the extract of the invention.

The present invention further provides a method for stimulating bone growth comprising the step of administering the composition of the invention containing the extract of crude drug complex to a target subject.

The target subject herein is preferably teenagers and dwarfism patients either congenital or acquired. The effective dosage of the extract of crude drug complex is preferably 0.0001-100 mg/kg per day, and more preferably 0.001-100 mg/kg per day. The administration frequency can be once a day or a few times a day. The above dosage cannot limit the scope of the invention in any way.

The extract of crude drug complex of the present invention can be administered by various pathways, for example the possible administration pathway can be oral administration, rectal administration, intravenous injection, intramuscular injection, hypodermic injection, intrauterine injection or intracerebroventricular injection.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### Example 1- Example 5

### Preparation of the Extract of Crude Drug Complex

Dipsaci radix, Astragalus membranaceus and Acanthopanax senticosus were purchased in Kyung-Dong market (Seoul, Korea) and dried. Each constituent was measured to meet the constituent ratio shown in Table 1 and pulverized. 70% ethanol was added thereto by 10 times the weight of the sample, followed by reflux heat extraction for 6 hours. The mixture was filtered under reduced pressure to separate the extract and residue. 70% ethanol (Examples 1-4) or water (Example 5) was added to the residue by 10 times the weight of the residue, followed by reflux heat extraction for 6 hours. The concentrated extract was obtained by concentrating the reactant with a rotary evaporator. The extract was freeze-dried at −70° C. to obtain powders. The yield of each herb powder was calculated. According to the proposed mixing ratio of each herb, the freeze-dried powder of each herb was mixed to prepare the final extract. This final extract was further used for the following experiments.

TABLE 1
Composition

| | | Dipsaci | Astragalus | Acanthopanax |
|---|---|---|---|---|
| | | radix | membranaceus | senticosus |
| Example 1 | Weight (g) | 40 | 20 | 0 |
| | Weight part | 2 | 1 | 0 |
| Example 2 | Weight (g) | 30 | 15 | 100 |
| | Weight part | 3 | 1.5 | 5 |
| Example 3 | Weight (g) | 15 | 30 | 100 |
| | Weight part | 1.5 | 3 | 5 |
| Example 4 | Weight (g) | 37.5 | 22.5 | 37.5 |
| | Weight part | 5 | 3 | 5 |
| Example 5 | Weight (g) | 30 | 15 | 100 |
| | Weight part | 3 | 1.5 | 5 |

### Comparative Examples 1 and 2

### Preparation of the Extract of each Herb

The composition composed by the ratio shown in Table 2 was extracted by the same manner as described in Examples 1-4. To compare the growth simulating effect, the positive control group was treated with growth hormone and the negative control group was administered with saline.

TABLE 2
Composition

| | | Dipsaci | Astragalus | Acanthopanax |
|---|---|---|---|---|
| | | radix | membranaceus | senticosus |
| Comparative | Weight | 145 | 0 | 0 |
| Example 1 | (g) | | | |
| | Weight | 1 | 0 | 0 |
| | part | | | |
| Comparative | Weight | 0 | 145 | 0 |
| Example 2 | (g) | | | |
| | Weight | 0 | 1 | 0 |
| | part | | | |
| Comparative | Weight | 0 | 0 | 145 |
| Example 3 | (g) | | | |
| | Weight | 0 | 0 | 1 |
| | part | | | |

### Experimental Example 1

### Growth Stimulated by the Extract of Crude Drug Complex

To measure the growth stimulated by the extract of crude drug complex, Sprague-Dawley line male rats (3 weeks of age) were purchased from Dae Han Bio Link Co (Korea) and used for the following experiment. The experimental procedure followed the guideline for animal management of NIH, USA. Equal conditions were given to each group, for example temperature was 20±2° C. and the light was given from 07:00 to 19:00. Food and water were provided by libitum and mice were weighed every day.

The rats were divided into 4 groups, 10 per each group. To the negative control group, saline was orally administered twice a day for 4 days. To the experimental group, 100 mg/kg of the extract of crude drug complex (Examples 1-5 and Table 1) or 100 mg/kg of the extract of each herb medicine (Comparative Examples 1-3 and Table 2) was orally administered likewise. To the positive control group, 20 μg/kg of growth hormone was hypodermically injected. On the 3^{rd} day, tetracycline (10 mg/kg) was injected into the abdominal cavity to deposit on the growth plate. On the 5^{th} day, after 48 hours from the tetracycline injection, the rats were anesthetized with ether and sacrificed. The growth was investigated after tetracycline was deposited on the growth plate by measuring the extended length of tibia, which was determined by measuring the interval between the growth plate and the luminescent line developed by the deposit of tetracycline.

The tibia was separated from the rat sacrificed on the 5^{th} day of experiment and fixed in 4% phosphate-buffered paraformaldehyde for 48 hours, followed by precipitating in 30% sucrose solution and dehydration. The fixed bone tissues were freeze-dried and sagital sections of tibia proximal part was cut by cryocut by 40 μm, resulting in the preparation of tissue samples. The sections were used for the bone growth analysis.

To investigate bone growth, the interval between the growth plate and the luminescent line developed by the deposition of tetracycline was observed under fluorescent microscope (Hansson et al., Calcified Tissue Research, 10(3), 238, 1972, FIG. 1) and the average of each section was calculated. Every measurement was performed by two observers by blind method. The results are represented by average±standard deviation. Student-t test was performed to distinguish the results among groups and Kruskall-Wallis test was also performed when inspection was not completely trustworthy in spite of the acknowledgement of significant difference of standard deviations.

As a result, the extract of crude drug complex stimulated bone growth. The bone growth rate of the experimental group treated with the extract of crude drug complex (Examples 1-5 and Table 1) was statistically higher than those of the negative control group and the group treated with the single extract of each herb (Comparative Example 1-3 and Table 2) and also higher than that of the positive control group injected with growth hormone. The bone growth rate of the group treated with the single extract of each herb (Comparative Example 1-3 and Table 2) was higher than that of the negative control group but lower than that of the positive control (Table 3 and FIG. 2). FIG. 2 illustrates the growth of each negative control, positive control (Table 3), and those of Comparative Examples 1-3 and Examples 1-3. * indicates P<0.01, and indicates indicates P<0.001.

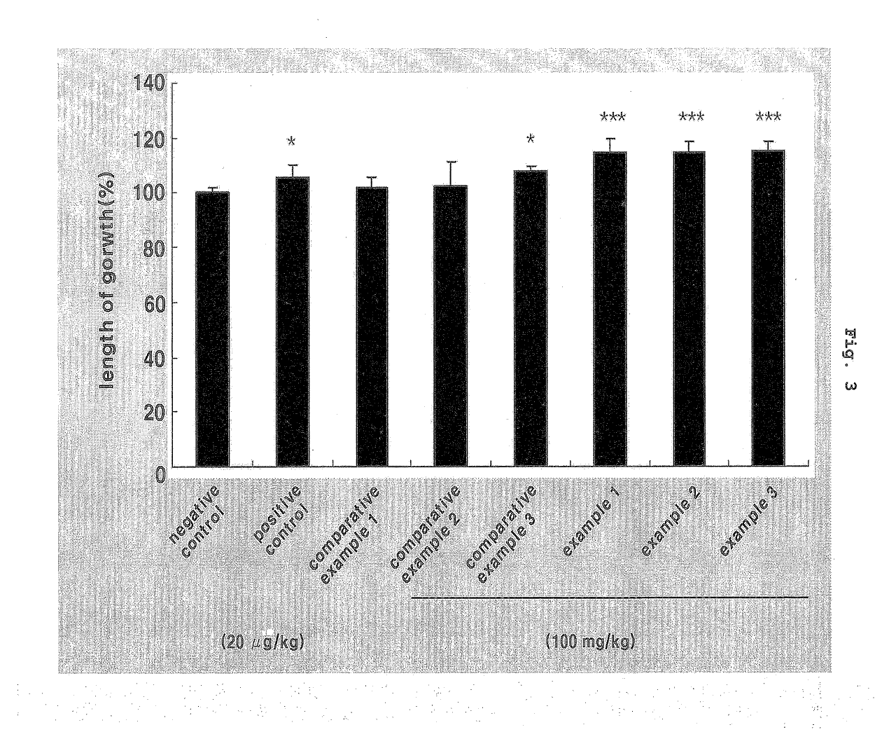

Considering the bone growth rate of the negative control treated with saline as 100%, the growth rates of the experimental group treated with the extract of crude drug complex (Examples 1-5 and Table 1), the group treated with the extract of each herb (Comparative Example 1-3 and Table 2) and the positive control were calculated. As a result, the growth rate of the experimental group treated with the extract of crude drug complex (Examples 1-5 and Table 1) was significantly higher than those of the negative control and the group treated with each herb extract (Comparative Example 1-3 and Table 2) and even higher than that of the positive control. In the meantime, the growth rate of the group treated with each herb extract (Comparative Example 1-3 and Table 2) was higher than that of the negative control but not than those of the positive control and the experimental group treated with the extract of crude drug complex (Examples 1-5 and Table 1). FIG. 3 illustrates the growth rates of the negative control, positive control, and those of Comparative Examples 1-3 and Examples 1-3. * indicates P<0.01, and *** indicates P<0.001.

TABLE 3
Growth and growth rate

| | Growth (μm/day) | Growth rate (%) |
|---|---|---|
| Negative control | 290.90 ± 6.23 | 100 ± 2.14 |
| (saline) | | |
| Positive control | 308.24 ± 12.15* | 105.96 ± 4.17* |
| (growth hormone) | | |
| Example 1 | 333.80 ± 15.03*** | 114.74 ± 5.17*** |
| Example 2 | 334.5 ± 12.49*** | 114.99 ± 3.78*** |
| Example 3 | 335.2 ± 11.78*** | 115.23 ± 3.57*** |
| Example 4 | 332.40 ± 18.31 | 114.27 ± 4.74 |
| Example 5 | 329.98 ± 15.82 | 113.43 ± 4.10 |
| Comparative Example 1 | 297.01 ± 11.53 | 102.10 ± 3.97 |
| Comparative Example 2 | 298.08 ± 26.29 | 102.55 ± 9.04 |
| Comparative Example 3 | 314.00 ± 14.08 | 114.75 ± 5.17 |

Statistical evaluation: student-T test
*P < 0.01
***P < 0.001
¹⁾Calculation formula = 100 + (growth of experimental group − growth of negative control)/growth of negative control × 100

### Experimental Example 2

### Acute Toxicity Test for the Extract of Crude Drug Complex

The following experiment was performed to see if the extract of crude drug complex had acute toxicity in mice.

4 week old SPF (specific pathogens free) ICR line mice were divided into 4 groups (6 mice per group, 3 female and 3 male each). The animals were raised in the animal laboratory at the temperature of 22±3° C. with the humidity of 55±10% under the light condition of 12L/12D. The mice were adapted for about 1 week before experiment. Feed (for mouse and rat, Dyets, USA) and water were sterilized and provided freely.

The extract of crude drug complex prepared in the above example was suspended in 0.5% tween 80 at the concentration of 50 mg/ml, which was orally administered once to mice by different concentrations of 0.04 ml per 20 g of weight (100 mg/kg), 0.2 ml per 20 g of weight (500 mg/kg) and 0.4 ml per 20 g weight (1,000 mg/kg). Side effects or death were observed for 7 days from the administration. Particularly, any symptoms and death were observed 1, 4, 8, and 12 hours later on the first day of administration and at least once in the morning and once in the afternoon from the next day to the 7^{th} day. On the 7^{th} day, the animals were sacrificed and any abnormal signs in the gastrointestinal organs of chest and abdomen were checked with the naked eye during autopsy. Weight changes were recorded every day from the first day of administration to observe weight loss by the extract of crude drug complex.

The results showed that the test samples did not cause any specific clinical symptoms, weight change, or death in mice. No change was observed in hematological tests, biochemical tests of blood, and autopsy.

Therefore, the extract of crude drug complex used in this experiment was evaluated to be a safe substance since it did not cause any toxic change in mice up to the level of 1,000 mg/kg and its estimated LD₅₀ value was much greater than 1,000 mg/kg in mice.

The Manufacturing Examples of the composition for the present invention are described hereinafter.

### Manufacturing Example 1

### Preparation of Pharmaceutical Formulations

### <1-1> Preparation of Injectable Solution

| | |
|---|---|
| Extract of crude drug complex | 10 mg |
| Sodium metabisulphite | 3.0 mg |
| Methylparaben | 0.8 mg |
| Propylparaben | 0.1 mg |
| Sterilized distilled water | proper amount |

Injectable solutions were prepared by mixing all the above components, putting the mixture into 2 ml ampoules and sterilizing thereof by the conventional method for preparing injectable solutions.

### <1-2> Preparation of Tablets

| | |
|---|---|
| Extract of crude drug complex | 200 mg |
| Starch | 100 mg |
| Lactose | 100 mg |
| Magnesium stearate | 2 mg |

Tablets were prepared by mixing all the above components by the conventional method for preparing tablets.

### <1-3> Preparation of Capsules

| | |
|---|---|
| Extract of crude drug complex | 10 mg |
| Lactose | 50 mg |
| Starch | 50 mg |
| Talc | 2 mg |
| Magnesium stearate | 2 mg |

Capsules were prepared by mixing all the above components, which filled gelatin capsules according to the conventional method for preparing capsules.

### <1-4> Preparation of Liquid Formulations

| | | |
|---|---|---|
| Extract of crude drug complex | 1000 | mg |
| Sugar | 20 | g |
| Isomerized sugar | 20 | g |
| Talc | 2 | mg |

| | |
|---|---|
| Lemon flavor | proper amount |

Total volume was adjusted to be 1000 ml by adding purified water. Liquid formulations were prepared by mixing all the above components, putting the mixture into brown bottles and sterilizing thereof by the conventional method for preparing liquid formulations.

### Manufacturing Example 2

### Preparation of Food

### <2-1> Preparation of Health Food

| Extract of crude drug complex | 1000 | mg |
|---|---|---|
| Vitamin complex | proper amount | |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinic acid amide | 1.7 | mg |
| Folic acid | 50 | μg |
| Calcium pantothenate | 0.5 | mg |
| Minerals | proper amount | |
| Ferrous sulfate | 1.75 | mg |
| Zinc oxide | 0.82 | mg |
| Magnesium carbonate | 25.3 | mg |
| Potassium phosphate monobasic | 15 | mg |
| Potassium phosphate dibasic | 55 | mg |
| Potassium citrate | 90 | mg |
| Calcium carbonate | 100 | mg |
| Magnesium chloride | 24.8 | mg |

Vitamins and minerals were mixed according to the preferable composition rate for health food. However, the composition rate can be adjusted. The constituents were mixed according to the conventional method for preparing health food and then the composition for health food (ex. health candies, etc) was prepared according to the conventional method.

<2-2> Preparation of Flour Food 0.1-10.0 weight part of the extract of crude drug complex was added to the flour. Health enhancing foods such as bread, cake, cookies, crackers and noodles were prepared with the flour mixture according to the conventional method.

### <2-3> Preparation of Soups and Gravies

0.1-10.0 weight part of the extract of crude drug complex was added to soups and gravies. Health enhancing meat products, soups and gravies were prepared with this mixture by the conventional method.

### <2-4> Preparation of Ground Beef

Health enhancing ground beef was prepared by mixing 10 weight part of the extract of crude drug complex with ground beef according to the conventional method.

### <2-5> Preparation of Dairy Products

0.1-1.0 weight part of the extract of crude drug complex was added to milk. Health enhancing dairy products such as butter and ice cream were prepared with the milk mixture according to the conventional method.

### <2-6> Preparation of Sun-Sik

Brown rice, barley, glutinous rice and Yulmu (Job's tears) were gelatinized according to the conventional method, dried and pulverized to obtain 60-mesh powders.

Black soybean, black sesame and wild sesame were steamed and dried according to the conventional method and pulverized to obtain 60-mesh powders.

The extract of crude drug complex was concentrated under reduced pressure, spray-dried and pulverized to obtain 60-mesh dry powders.

Sun-Sik was prepared by mixing the dry powders of the grains, seeds and the extract of crude drug complex according to the below ratio.

Grains (brown rice: 30 weight part, Yulmu: 15 weight part, barley: 20 weight part, glutinous rice: 10 weight part),

Seeds (wild sesame: 7 weight part, black soybean: 8 weight part, black sesame: 7 weight part),

Dry powders of the extract of crude drug complex (1 weight part),

Ganoderma lucidum (0.5 weight part),

Rehmannia glutinosa (0.5 weight part)

### Manufacturing Example 3

### Preparation of Beverages

### <3-1> Preparation of Health Beverages

| | | |
|---|---|---|
| Extract of crude drug complex | 1000 | mg |
| Citric acid | 1000 | mg |
| Oligosaccharide | 100 | g |
| Maesil (Prunus mume) Extract | 2 | g |
| Taurine | 1 | g |
| Purified water | up to 900 | ml |

The above constituents were mixed according to the conventional method for preparing health beverages. The mixture was heated at 85° C. for 1 hour with stirring and then filtered. The filtrate was loaded in 2 liter sterilized containers, which were sealed and sterilized again, stored in a refrigerator until they would be used for the preparation of a composition for health beverages.

The constituents appropriate for favorite beverages were mixed according to the preferred mixing ratio but the composition ratio can be adjusted according to regional and national preferences, etc.

### <3-2> Preparation of Vegetable Juice

Health enhancing vegetable juice was prepared by adding 0.5 g of the extract of crude drug complex to 1,000 ml of tomato or carrot juice according to the conventional method.

### <3-3> Preparation of Fruit Juice

Health enhancing fruit juice was prepared by adding 0.1 g of the extract of crude drug complex to 1,000 ml of apple or grape juice according to the conventional method.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A composition for stimulating bone growth containing an extract of a herb mixture comprising Dipsaci radix, Astragalus membranaceus and Acanthopanax senticosus as an active ingredient.

2. The composition for stimulating bone growth according to claim 1, wherein the extract is extracted from a herb mixture comprising 1-5 weight part (w/w) of Dipsaci radix, 1-3 weight part of Astragalus membranaceus and 1-5 weight part of Acanthopanax senticosus.

3. The composition for stimulating bone growth according to claim 1, wherein the extract is extracted using water, alcohol or the mixture thereof.

4. The composition for stimulating bone growth according to claim 3, wherein the alcohol is C₁-C₄ low alcohol.

5. The composition for stimulating bone growth according to claim 4, wherein the low alcohol is ethanol.

6. The composition for stimulating bone growth according to claim 1, wherein the extract is extracted at room temperature for 1-24 hours.

7. A health food for stimulating bone growth containing the composition of claim 1 as an active ingredient.

8. A method for stimulating bone growth comprising the step of administering a composition containing an extract of a herb mixture comprising Dipsaci radix, Astragalus membranaceus and Acanthopanax senticosus as an active ingredient to a target subject.

9. The method for stimulating bone growth according to claim 8, wherein the target subject is a teenager or dwarfism patient.

10. The method for stimulating bone growth according to claim 8, wherein the extract is extracted from a herb mixture comprising 1-5 weight part (w/w) of Dipsaci radix, 1-3 weight part of Astragalus membranaceus and 1-5 weight part of Acanthopanax senticosus.

11. The method for stimulating bone growth according to claim 8, wherein the extract is extracted using water, alcohol or the mixture thereof.

12. The method for stimulating bone growth according to claim 11, wherein the alcohol is C₁-C₄ low alcohol.

13. The method for stimulating bone growth according to claim 12, wherein the low alcohol is ethanol.

14. The method for stimulating bone growth according to claim 8, wherein the extract is extracted at room temperature for 1-24 hours.
